(19) United States
(12) Patent Application Publication
(10) Pub. No.: US 2010/0267760 A1
(43) Pub. Date: Oct. 21, 2010

(54) Methods of and Combination Therapies for Treating or Delaying the Onset of Dyskinesia

(76) Inventors: Gilberto Fisone, Stockholm (SE); Emmanuel Valjent, Stockholm (SE); Emanuela Santini, New York, NY (US)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/746,534

(22) PCT Filed: Dec. 5, 2008
(86) PCT No.: PCT/IB2008/055125

§ 371 Date: Jun. 9, 2010

Related U.S. Application Data
(60) Provisional application No. 61/012,105, filed on Dec. 7, 2007.

(51) Int. Cl. A61K 31/436; A61P 25/16; A61P 1/06

(52) U.S. Cl. 514/291

(57) ABSTRACT

Methods of treating or delaying onset of levodopa-induced dyskinesia in an individual comprise administering to the individual an effective amount of mammalian target of rapamycin (mTOR) inhibitor. Methods of treating or delaying onset of dyskinesia in an individual, wherein the dyskinesia is induced by administration of a drug causing abnormal protein expression in striatal medium-size spiny neurons (MSNs), comprise administering to the individual an effective amount of mTOR inhibitor. Further, a combination therapy for an individual having Parkinson's Disease comprises levodopa or a precursor or functional analogue thereof and mTOR inhibitor.

### RELATED APPLICATIONS

The present application claims priority under 35 U.S.C. §119 of U.S. Application Ser. No. 61/012,105 filed Dec. 7, 2007.

### FIELD OF THE INVENTION

The present invention is directed to methods of treating or delaying the onset of drug-induced dyskinesia in an individual with, for example, Parkinson's Disease, by administering mammalian target of rapamycin (mTOR) inhibitor. Such dyskinesia is typically induced by administration of levodopa, which is commonly employed for treatment of Parkinson's Disease. The invention is also directed to combination therapies comprising levodopa or a precursor or functional analogue thereof and mTOR inhibitor.

### BACKGROUND OF THE INVENTION

Dyskinesia involves abnormal involuntary movements (AIMs) and, more specifically, impairment in the ability to control movements, characterized by spasmodic or repetitive motions or lack of coordination, and is typically generated by prolonged administration of the drug levodopa. As levodopa is the most common treatment for Parkinson's Disease, dyskinesia is one of the major challenges facing the current therapy for Parkinson's Disease (Obeso et al, Trends Neurosci., 23:S2-7 (2000)). The debilitating motor disturbances of dyskinesia are all the more problematic because levodopa, in spite of its introduction several decades ago, still represents the therapy of choice for the treatment of Parkinson's disease. Moreover, dyskinesia is clearly manifested in patients even after transplantation with fetal mesencephalic tissue (Olanow et al, Ann. Neurol., 54:403-414 (2003)).

The glutamate antagonist amantadine has been described as an efficacious drug in the treatment of levodopa-induced dyskinesia (LID). The use of amantadine, however, is complicated by side effects, such as confusion, worsening of hallucinations and edema (Fabbrini et al, Mov. Disord., 22:1379-1389 (2007)). The discovery of pharmacological interventions able to counteract LID would therefore represent an important breakthrough in the therapy for Parkinson's Disease.

The therapeutic efficacy of levodopa is based on its conversion to dopamine, which re-establishes normal neurotransmission in the Parkinsonian brain. The main target of levodopa is the GABAergic medium-size spiny neuron (MSN) of the dorsal striatum, which has lost most of its dopaminergic innervation following degeneration of substantia nigra neurons. MSNs represent the vast majority of neurons present in the striatal formation and are critically involved in the control of motor function. A large number of studies showed that changes in the activity of molecules involved in signal transduction at the level of MSNs play important roles in the generation of various types of motor responses. More recently, specific alterations in MSN signal transduction have been linked to the expression of LID. Abnormal activation of the extracellular signal-regulated kinases 1 and 2 (ERK) has been associated to LID and the pharmacological blockade of ERK has been shown to counteract the development of dyskinesia (Santini et al, J. Neurosci., 27:6995-7005 (2007)). However, drugs blocking ERK activation are likely to affect basic physiological processes and are therefore poorly suitable as clinical agents. It is therefore important to identify targets located downstream of ERK activation in order to increase the specificity of intervention and avoid major side effects.

Accordingly, methods and/or therapies for treating or delaying onset of drug-induced dyskinesia such a levodopa-induced dyskinesia are desired.

### SUMMARY OF THE INVENTION

It is an object of the present invention to provide methods and therapies for treating or delaying onset of drug-induced dyskinesia such a levodopa-induced dyskinesia.

In one embodiment, the present invention is directed to a method of treating or delaying onset of levodopa-induced dyskinesia in an individual. The method comprises administering to the individual an effective amount of mammalian target of rapamycin (mTOR) inhibitor.

In another embodiment, the invention is directed to a method of treating or delaying onset of dyskinesia in an individual, wherein the dyskinesia is induced by administration of a drug causing abnormal protein expression in striatal medium-size spiny neurons (MSNs). The method comprises administering to the individual an effective amount of mammalian target of rapamycin (mTOR) inhibitor.

In yet another embodiment, the invention is directed to a combination therapy for an individual having Parkinson's Disease, comprising levodopa or a precursor or functional analogue thereof and mammalian target of rapamycin (mTOR) inhibitor.

The methods and combination therapies of the invention are advantageous in providing treatment for dyskinesia and/or delaying onset of dyskinesia while avoiding significant adverse side effects. These and additional objects and advantages of the present invention will be more fully understood in view of the following detailed description.

### BRIEF DESCRIPTION OF THE DRAWINGS

Various aspects of the invention and the detailed description will be more apparent in view of the drawings in which:

FIG. 1 presents a simple regression analysis showing a significant correlation between LID, determined as abnormal involuntary movements (AIMs), and mTOR-dependent phosphorylation of (a) p70S6K, and (b) ribosomal protein S6. The levels of phospho-Thr389-p70S6K and phospho-Thr240/244-S6 were determined by western blotting as described in the Example in 6-OHDA-lesioned mice treated for 10 days with 20 mg/kg of L-DOPA and expressed as percentage of those measured in control mice (Sham-lesioned treated with saline). The broken line indicates the median value (28.5) of total AIMs scores. (a) R=0.76 (p<0.0001) and (b) R=0.81 (p<0.0001). Upper panels show representative western blotting obtained from the striata of a Sham-lesioned mouse (Sham), a high dyskinetic mouse (H Dys) and low dyskinetic mouse (L Dys).

FIG. 2 presents a simple regression analysis showing a significant correlation between LID, determined as abnormal involuntary movements (AIMs), and phosphorylation of 4E-BP. The levels of phospho-Ser65-4E-BP were determined by western blotting as described in the Example in 6-OHDA-lesioned mice treated for 10 days with 20 mg/kg of L-DOPA and expressed as percentage of those measured in control mice (Sham-lesioned treated with saline). The broken line indicates the median value (28.5) of total AIMs scores. R=0.83 (p<0.0001). Upper panels show representative western blotting obtained from the striata of a Sham-lesioned mouse (Sham), a high dyskinetic mouse (H Dys) and low dyskinetic mouse (L Dys).

FIG. 3 shows that rapamycin blocks the mTOR-dependent increase in S6 and 4E-BP phosphorylation produced by activation of dopamine D1 receptors, without affecting mTOR-independent signaling. Striatal slices from sham-, or 6-OHDA-lesioned mice as described in the Example were incubated for 5 min with vehicle, SKF81297 (a D1 receptor agonist) and SKF81297 plus rapamycin as indicated. The levels of phospho-S6 and phospho-4E-BP, phospho-ERK2 and phospho-GluR1 were determined by western blotting as described in the Example. Upper panels show representative autoradiograms. Lower panels represent means±SEM (n=3-6). Note the absence of rapamycin effect on SKF81297-induced phosphorylation of ERK2 and GluR1, which are not regulated by mTOR (two panels on the right). † P<0.01 vs. Sham/vehicle group, * P<0.01 and *** P<0.0001 vs. 6-OHDA/SKF81297; one-way ANOVA and Bonferroni test.

FIG. 4 shows that systemic administration of rapamycin blocks the mTOR-dependent increase in S6 phosphorylation produced, in striatal MSNs, by L-DOPA, without affecting ERK activation. Sham-, or 6-OHDA-lesioned mice as described in the Example were treated with L-DOPA/benserazide alone or in combination with rapamycin. Immunofluorescent detection is shown for S6 (a) and ERK (c) phosphorylation in the dorsal striata of a sham-lesioned mouse treated with L-DOPA and of 6-OHDA-lesioned mice treated with L-DOPA or L-DOPA+rapamycin. Quantification is shown of P-S6 (b) and P-ERK (d) positive cells in the dorsal striatum of sham- and 6-OHDA-lesioned mice treated with L-DOPA alone, or in combination with rapamycin. Data are expressed as means±SEM (n=3). ††\ P<0.0001 vs. Sham/L-DOPA and ***P<0.0001 vs. 6-OHDA/L-DOPA; one-way ANOVA and Bonferroni test.

FIG. 5 shows that systemic administration of rapamycin blocks the mTOR-dependent increase in 4E-BP phosphorylation produced by L-DOPA. The levels of phospho-Ser65-4E-BP were determined by western blotting as described in the Example in 6-OHDA-lesioned mice treated for 10 days with 20 mg/kg of L-DOPA and expressed as percentage of those measured in control mice (Sham-lesioned and treated with saline). The upper panel shows a representative western blot obtained from the striata of a Sham-lesioned mouse (Sham), a mouse treated with L-DOPA and a mouse treated with L-DOPA plus rapamycin. Lower panel represents means±SEM (n=4-8). One-way ANOVA indicated a significant effect of the treatment [F(2,13)=14.2, P<0.001]. *** p<0.001 vs Sham/Dopa and †† p<0.01 vs 6-OHDA/DOPA, Bonferroni test.

### DETAILED DESCRIPTION

The methods and combination therapies of the invention are advantageous in providing treatment for dyskinesia and/or delaying onset of dyskinesia.

Several lines of evidence indicate that prolonged administration of levodopa alters the functioning of striatal MSNs through long-term adaptive changes involving transcriptional (DNA to mRNA) and/or translational (mRNA to proteins) regulation of selected gene products. The inventors recently found that the mammalian target of rapamycin (mTOR) signaling pathway, which regulates translational efficiency of specific mRNAs, is profoundly altered in LID. This pathway is involved in the regulation of the formation of eIF4F, the initiation complex required for translation. eIF4F formation depends on the availability of the initiation factor eIF4E, which is normally sequestered by the inhibitory binding protein 4E-BP. Phosphorylation of 4E-BP catalyzed by the mTOR promotes initiation of translation via dissociation of the eIF4E/4E-BP complex (Proud, Biochem. J., 403:217-234 (2007)). mTOR also phosphorylates the p70S6 kinase (p70S6K), which most likely leads to activation of translation, via phosphorylation of the ribosomal protein S6. Based on this evidence, mTOR is generally regarded as a key factor in the control of mRNA translation.

By examining the state of phosphorylation of proteins regulated by mTOR, it has now been demonstrated that an association exists between activation of mTOR signaling and LID, and the present discovery provides the molecular framework for the development of antidyskinetic drugs able to block abnormal protein expression occurring in striatal MSNs by selectively interfering with translational efficiency rather than with general transcriptional and/or translational activity. Particularly, by administration of mTOR inhibitor, thereby inhibiting mTOR dependent signaling, LID is reduced and/or its onset is delayed, i.e., prevented. Accordingly, in one embodiment, the invention is directed to methods of treating or delaying onset of levodopa-induced dyskinesia in an individual comprise administering to the individual an effective amount of mTOR inhibitor. In a specific embodiment, the individual has Parkinson's Disease and the levodopa is administered for treatment of the Parkinson's Disease. In another embodiment, the invention is directed to methods of treating or delaying onset of dyskinesia in an individual, wherein the dyskinesia is induced by administration of a drug causing abnormal protein expression in striatal medium-size spiny neurons (MSNs), i.e., drugs which, like levodopa, target the GABAergic MSNs of the dorsal striatum. The method comprises administering to the individual an effective amount of mammalian target of rapamycin mTOR inhibitor. In a further embodiment, the invention is directed to a combination therapy for an individual having Parkinson's Disease, comprising levodopa or a precursor or functional analogue thereof and mTOR inhibitor.

The term “mTOR inhibitor” as used herein includes, but is not limited to, rapamycin (sirolimus) or a derivative thereof, such as temsirolimus (CCI-779, Wyeth), everolimus (RAD-001, Novartis Pharma AG) and AP-23573 (Ariad Pharmaceuticals), and the mTOR inhibitor, TAFA93 (Isotechnica Inc.). Rapamycin is a known macrolide antibiotic produced by Streptomyces hygroscopicus. Suitable derivatives of rapamycin include not only the compounds set forth above, but generally, compounds of formula A

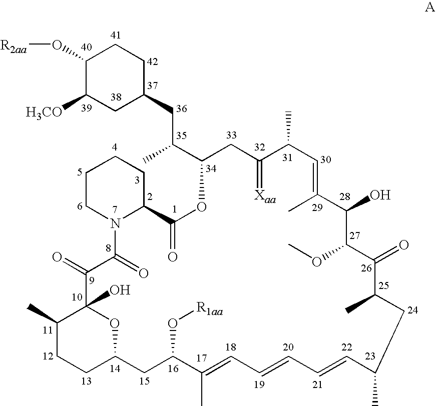

wherein R₁ₐₐ is CH₃ or C₃₋₆ alkynyl, R₂ₐₐ is H or —CH₂—CH₂—OH, 3-hydroxy-2-(hydroxymethyl)-2-methyl-propanoyl or tetrazolyl, and Xₐₐ is ═O, (H,H) or (H,OH), provided that R₂ₐₐ is other than H when Xₐₐ is ═O and R₁ₐₐ is CH₃ or a prodrug thereof when R₂ₐₐ is —CH₂—CH₂—OH, e.g. a physiologically hydrolysable ether thereof, e.g. a compound wherein R₂ₐₐ is —CH₂—CH₂—O-Alk, Alk being a C₁₋₉ alkyl optionally interrupted in the chain by 1 or 2 oxygen atoms. Compounds of formula A are disclosed, e.g. in WO 94/09010, WO 95/16691, WO 96/41807, U.S. Pat. No. 5,362,718 or WO 99/15530 which are incorporated herein by reference. They may be prepared as disclosed or by analogy to the procedures described in these references. Further examples of rapamycin derivatives include, e.g., ABT578 or 40-(tetrazolyl)-rapamycin, particularly 40epi-(tetrazolylrapamycin), e.g. as disclosed in WO 99/15530, and the so-called rapalogs, e.g. as disclosed in WO 98/02441, WO 01/14387 and WO 03/64383, e.g., AP23464, AP23675 or AP23841. Further examples of rapamycin derivatives are those disclosed under the names TAFA-93, biolimus-7 and biolimus-9.

The mTOR inhibitor is administered in an amount effective to reduce or delay onset of clinical indications of dyskinesia. In a specific embodiment, the mTOR inhibitor is administered in a dosage amount of from about 0.01 mg/kg to about 10 mg/kg. In another embodiment, the mTOR inhibitor is administered in a dosage amount of from about 0.1 mg/kg to about 8 mg/kg. In yet another embodiment, the mTOR inhibitor is administered in a dosage amount of from about 1 mg/kg to about 6 mg/kg. In another embodiment, the mTOR inhibitor is provided in a combination therapy with a drug causing abnormal protein expression in striatal medium-size spiny neurons (MSNs). In a more specific embodiment, the mTOR inhibitor is provided in a combination therapy with levodopa or a precursor or functional analogue thereof, for example in the treatment of Parkinson's Disease. In a yet more specific embodiment, the combination therapy employs the levodopa or precursor or functional analogue thereof in a dosage amount of from about 0.1 mg/kg to about 40 mg/kg.

The combination therapy may employ a single dosage form or multiple dosage forms wherein the mTOR inhibitor and the drug as described are administered simultaneously or sequentially, and, if sequentially, in immediate sequential administration or, alternatively, with a period of time between the respective administrations of, for example 1-12 or more hours therebetween. Thus, in a specific embodiment, the mTOR inhibitor and the levodopa or precursor or functional analogue thereof are provided in a single dosage form. In another specific embodiment, the mTOR inhibitor and the levodopa or precursor or functional analogue thereof are provided in separate dosage forms.

Advantageously, as will be demonstrated in the Example, the mTOR inhibitor blocks S6 and 4-E-BP phosphorylation increase produced by administration of levodopa, or other similarly acting drug, without affecting extracellular signal-regulated kinase (ERK) phosphorylation. Thus, the present methods and therapy reduce or prevent dyskinesia without producing significant adverse effects.

Additional advantages and aspects of the invention will be more apparent in view of the following Example which demonstrates various aspects of the invention.

### EXAMPLE

This Example employs a mouse model of LID to demonstrate the methods of the invention.

Materials and Methods

Animals. Male C57BL/6 mice (30 g) were purchased from Charles-River Laboratories (Sulzfeld, Germany). All the studies were performed in accordance with the Swedish Animal Welfare Agency.

Drugs. L-DOPA (methyl-L-DOPA hydrochloride; 20 mg/kg) and the peripheral DOPA decarboxylase inhibitor, benserazide hydrochloride (12 mg/kg), (Sigma Aldrich AB, Stockholm, Sweden) were dissolved in physiological saline immediately before use and injected i.p. in a volume of 10 ml/kg of body weight. The mTOR inhibitor Rapamycin (5 mg/kg) was dissolved in a mixture of 5% tween-20, 5% DMSO and 15% PEG500, in H₂O.

6-OHDA lesion. C57B1/6 mice were anaesthetized with a mixture of fentanyl citrate (0.315 mg/ml), fluanisone (10 mg/ml) (VetaPharma, Leeds, UK), midazolam (5 mg/ml) (Hameln Pharmaceuticals, Gloucester, UK) and water (1:1:2; in a volume of 2.7 ml/kg), and mounted in a stereotactic frame (David Kopf Instruments, Tujunga, Calif.) equipped with a mouse-adaptor. 6-OHDA-HCl (Sigma Aldrich AB, Stockholm, Sweden) was dissolved in 0.02% ascorbic acid in saline at the concentration of 3.0 μg of freebase 6-OHDA/μl. Mice received unilateral injections (2×2 μl) of vehicle or 6-OHDA into the right striatum at the following coordinates according to the mouse brain atlas (Paxinos et al, The Rat Brain in Stereotaxic Coordinates, New York, Academic Press (1982)): anteroposterior +1.0 mm, mediolateral −2.1 mm, dorsoventral −3.2 mm; anteroposterior +0.3 mm, mediolateral −2.3 mm, dorsoventral −3.2. Each injection was performed at a rate of 0.5 μl/min using a glass capillary with an outer diameter of approximately 50 μm attached to a 10 μl Hamilton syringe. Following the injection, the capillary was left in place for an additional 3 min before slowly retracting it (Santini et al, supra). Mice were allowed to recover for four weeks, before behavioural evaluation and drug treatment. Lesions were assessed at the end of the experiments by determining the striatal levels of tyrosine hydroxylase (TH) using western blotting (see below).

Determination of LID. 6-OHDA-lesioned mice were treated for 10 days with one injection per day of L-DOPA (20 mg/kg) plus benserazide (12 mg/kg). AIMs were assessed following the last injection of L-DOPA, using a previously established and validated mouse model of LID (Lundblad et al, Neurobiol. Dis., 16:110-123 (2004) and Lundblad et al, Exp. Neurol., 194:66-75 (2005)). Briefly, 20 min following L-DOPA administration, mice were placed in separate cages and individual dyskinetic behaviours were assessed for 1 min (monitoring period) every 20 min, over a period of 140 min. Purposeless movements, clearly distinguished from natural stereotyped behaviours (i.e. grooming, sniffing, rearing, and gnawing), were classified into four different subtypes: locomotive AIMs (tight contralateral turns), axial AIMs (contralateral dystonic posture of the neck and upper body toward the side contralateral to the lesion), limb AIMs (jerky and fluttering movements of the limb contralateral to the side of the lesion), orolingual AIMs (vacuous jaw movements and tongue protrusions). Each subtype was scored on a severity scale from 0 to 4, where 0=absent, 1=occasional, 2=frequent, 3=continuous, 4=continuous and not interruptible by outer stimuli.

Tissue extraction. Twenty-four hr after AIMs assessment, the mice were treated with L-DOPA/benserazide and killed by decapitation 30 min later. The heads of the animals were immediately immersed in liquid nitrogen for 6 seconds. The brains were then removed and the striata were dissected out within 20 seconds on an ice-cold surface, sonicated in 750 μl of 1% sodium dodecylsulfate and boiled for 10 minutes. The effectiveness of this extraction procedure in preventing protein phosphorylation and dephosphorylation, hence ensuring that the level of phosphoproteins measured ex vivo reflects the in vivo situation, has previously been tested, (Svenningsson et al, Proc. Natl. Acad. Sci. USA, 97:1856-1860 (2000)). Aliquots (5 μl) of the homogenate were used for protein determination using a BCA (bicinchoninic acid) assay kit (Pierce Europe, Oud Beijerland, the Netherlands).

Western blotting. Equal amounts of protein (30 μg) for each sample were loaded onto 10% polyacrylamide gels. Proteins were separated by sodium dodecylsulfate-polyacrylamide gel electrophoresis and transferred overnight to (PVDF) membranes (Amersham Pharmacia Biotech, Uppsala, Sweden) (Towbin et al, Proc. Natl. Acad. Sci. USA, 76:4350-4354 (1979)). The membranes were immunoblotted using phospho-Thr202/Tyr204-ERK1/2, phosphoThr389-p70S6K, phosphoSer240/244-S6 ribosomal protein and phosphoSer65-4E-BP (Cell Signaling Technology, Beverly, Mass.) and phospho-Ser845-GluR1 (PhosphoSolutions, Aurora, Colo.). Antibodies against p70S6K, S6 ribosomal protein and 4E-BP ERK1/2 and GluR1 (Cell Signaling Technology, Beverly, Mass.) that are not phosphorylation state specific were used to estimate the total amount of proteins. Detection was based on fluorescent secondary antibody binding detected and quantitated using a Li-Cor Odyssey infrared fluorescent detection system (Li-Cor, Lincoln, Nebr.). The levels of each phosphoprotein were normalized for the amount of the corresponding total protein detected in the sample.

Preparation and incubation of striatal slices. Sham, or 6-OHDA-lesioned C57B1/6 mice (25-30 g) were killed by decapitation, and the brains were rapidly removed. Coronal slices (250 μm) were prepared using a vibroslice (Leica Microsystems, Nussloch, Germany). Dorsal striata were then dissected out from each slice under a microscope. Two slices were placed in individual 5-ml polypropylene tubes containing 2 ml of Krebs-Ringer's bicarbonate buffer (KRB; 118 mM NaCl, 4.7 mM KCl, 1.3 mM CaCl₂, 1.5 mM MgSO₄, 1.2 mM KH₂PO₄, 25 mM NaHCO₃ and 11.7 mM glucose, equilibrated with 95% O₂/5% CO₂ (vol/vol), pH 7). The samples were equilibrated at 30° C. for two 30-min intervals, each followed by replacement of the medium with 2 ml fresh KRB. Slices were incubated for 5 min in the presence of vehicle, SKF81297, or SKF81297 plus rapamycin. After incubation, the solutions were rapidly removed, the samples were sonicated in 100 μl of 1% sodium dodecyl sulfate and boiled for 10 min. Aliquots (5 μl) of the homogenate were used for protein content determination before western blotting assay.

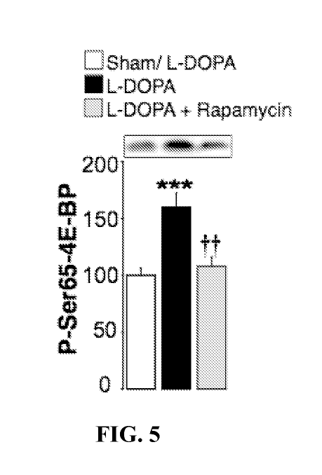

Tissue preparation and immunofluorescence. Sham, or 6-OHDA-lesioned mice were pre-treated for 4 days with 5 mg/kg of rapamycin (one injection per day). On the 4th day, mice received L-DOPA (20 mg/kg in combination with 12 mg/kg of benserazide) 1 hr after the last administration of rapamycin. For the determination of phospho-Ser65-4E-BP (cf. FIG. 5), the mice were killed by decapitation 30 min after L-DOPA administration and the tissue extracted for western blotting analysis (see above description). For the determination of phospho-Ser240/244-56 and phospho-Thr202/Tyr204-ERK1/2 (cf. FIG. 4), mice were rapidly anaesthetized with pentobarbital (50 mg/kg, i.p., Sanofi-Aventis, France) 30 min after L-DOPA administration and perfused transcardially with 4% (weight/vol) paraformaldehyde in 0.1 M sodium phosphate buffer (pH 7.5). Brains were post-fixed overnight in the same solution and stored at 4° C. Thirty μm-thick sections were cut with a vibratome (Leica, Nussloch, Germany) and stored at −20° C. in 0.1 M sodium phosphate buffer containing 30% (vol/vol) ethylene glycol and 30% (vol/vol) glycerol. Free-floating sections were rinsed in Tris-buffered saline (TBS; 0.25 M Tris and 0.5 M NaCl, pH 7.5), incubated for 5 min in TBS containing 3% H₂O₂ and 10% methanol (vol/vol), and then rinsed three times for 10 min each in TBS. After 20 min incubation in 0.2% Triton X-100 in TBS, sections were again rinsed three times in TBS. Finally, they were incubated overnight at 4° C. with the different primary antibodies. For detection of phosphorylated proteins, 0.1 mM NaF was included in all buffers and incubation solutions. Phosphorylation of proteins was analyzed using rabbit polyclonal antibodies against phospho-Thr202/Tyr204-ERK1/2 and phospho-Ser240/244-S6 ribosomal protein (Cell Signaling Technology, Beverly, Mass.). Following incubation with primary antibodies, sections were rinsed three times for 10 min in TBS and incubated for 45 min with goat Cy3-coupled secondary antibody (Jackson Laboratory, Bar Harbor, Me.). Sections were rinsed for 10 min twice in TBS and twice in TB (0.25 M Tris) before mounting in 1,4-Diazabicyclo-[2.2.2]-octane (DABCO, Sigma-Aldrich Sweden AB). Single-labeled images from dorsal striatum were obtained bilaterally using sequential laser scanning confocal microscopy (Zeiss LSM). Neuronal quantification was performed in 562×562 μm images by counting Cy3 immunofluorescence.

Results

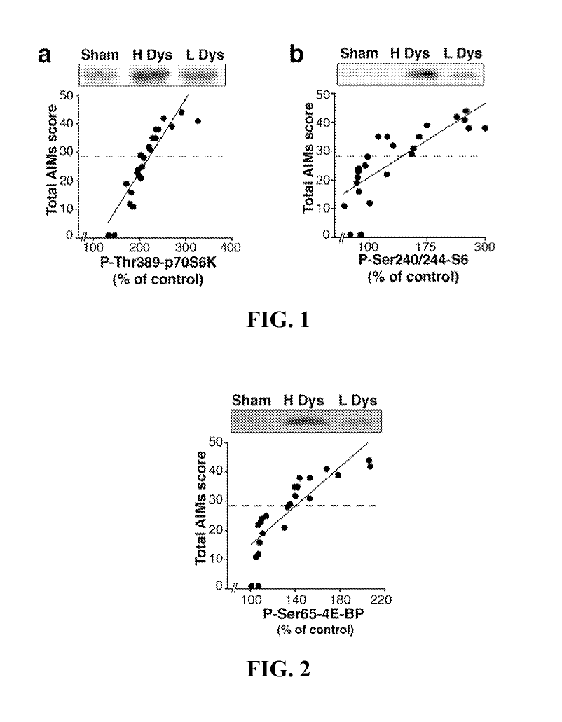

The activity mTOR is examined by determining the state of phosphorylation of the mTOR substrates p70S6K and 4E-BP. FIG. 1 presents a simple regression analysis showing the significant correlation between LID, determined as abnormal involuntary movements (AIMs) as described, and mTOR-dependent phosphorylation of (a) p70S6K, and (b) ribosomal protein S6. FIG. 1 shows that phosphorylation of p70S6K at Thr389, a site specifically regulated by mTOR, correlates with LID (FIG. 1a) and that activation of p70S6K is accompanied by increased phosphorylation of its primary substrate, the ribosomal protein S6 (FIG. 1b). Taken together these data indicate that LID is associated to regulation of proteins involved in mRNA transcription.

The formation of the initiation of translation complex, eIF4F, depends on the dissociation of eIF4E from 4E-BP. This event is mediated by mTOR catalyzed phosphorylation of 4E-BP. FIG. 2 presents a simple regression analysis showing a significant correlation between LID, determined as abnormal involuntary movements (AIMs), and phosphorylation of 4E-BP and shows that the striata of highly dyskinetic mice contains higher levels of phospho-4E-BP, compared to low dyskinetic mice and that the levels of phosphorylated 4E-BP correlate with the severity of dyskinesia. These results further support the finding of increased mTOR signaling associated to LID.

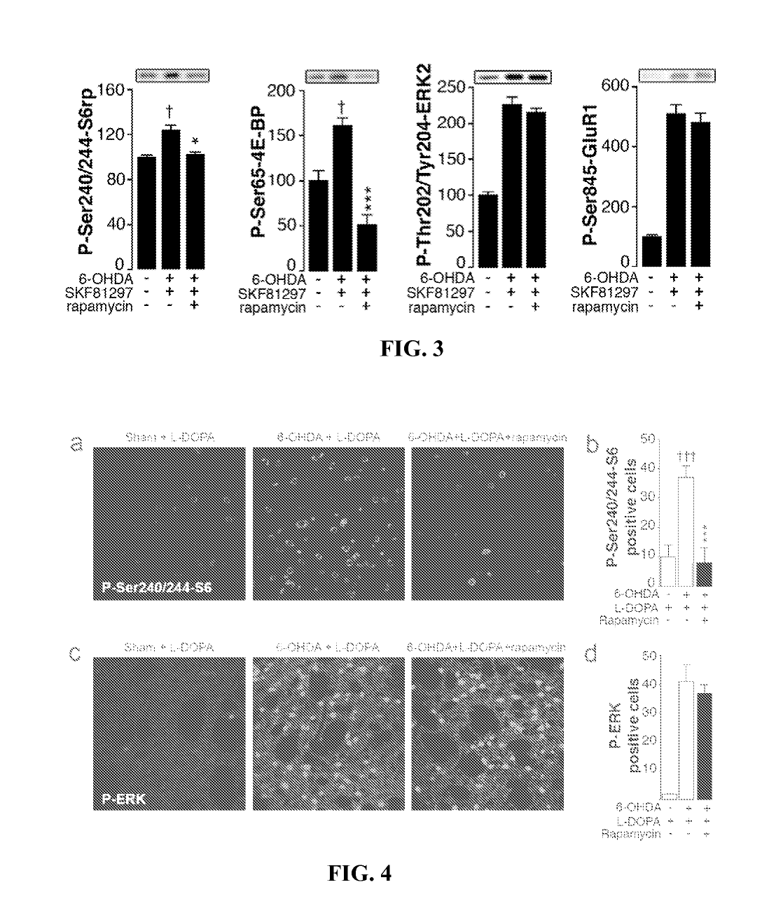

FIG. 3 shows that rapamycin blocks the mTOR-dependent increase in S6 and 4E-BP phosphorylation produced by activation of dopamine D1 receptors (which act similarly to L-DOPA), without affecting mTOR-independent signaling, while FIG. 4 shows that systemic administration of rapamycin blocks the mTOR-dependent increase in S6 phosphorylation produced by L-DOPA, without affecting ERK activation. FIG. 5 shows that rapamycin is also able to prevent the increase in 4E-BP phosphorylation produced by administration of L-DOPA. Specifically, rapamycin prevents the mTOR-dependent increase in phosphorylation of S6 and 4E-BP produced by SKF81297, without affecting ERK or cAMP signaling, as shown by lack of rapamycin effect on the phosphorylation of ERK and of the cAMP-dependent protein kinase regulated site of GluR1 as shown in the results in FIG. 3. FIGS. 4 and 5 show the efficacy of rapamycin in intact animals and particularly that rapamycin blocks the increase in S6 and 4E-BP phosphorylation produced by systemic administration of L-DOPA, without affecting ERK phosphorylation.

These results show that drugs able to inhibit mTOR dependent signaling are useful in the preventive and symptomatic treatment of LID. Because of the involvement of mTOR in the control of cell cycle, cell growth and proliferation, mTOR inhibitors have been developed as immunosuppressants and anticancer drugs and have been suggested for use in the treatment of neurodegenerative disorders. However, The mechanisms by which this class of drugs would exert neuroprotection are radically different from the mechanism employed in the present methods and therapies as antidyskinetic agents. First, the neuroprotective effect of mTOR inhibitors is exerted by acting on midbrain dopaminergic neurons, whereas the antidyskinetic effect is exerted by acting on GABAergic striatal neurons. Second, the downstream proteins regulated by mTOR and potentially responsible for neurodegeneration are most likely different from those possibly implicated in LID. In contrast, the mechanism at the basis of the antidyskinetic properties of mTOR inhibitors in the present methods and therapies is based on the ability of these compounds to prevent the development of LID by blocking maladaptive mechanisms occurring in striatal neurons (independently of possible neuroprotective effects exerted on dopaminergic neurons). The fact that suppression or reduction of mTOR signaling could block the abnormal effects produced by L-DOPA on striatal neurons indicates that mTOR inhibitors may be used not only in the preventive therapy for LID, but also as symptomatic agents in the treatment of patients who have already developed dyskinesia.

The methods and therapies of the present invention have been described with reference to specific embodiments and the Example demonstrates various specific aspects of the invention. However, it will be appreciated that additional embodiments, aspects, variations and modifications of the invention can be effected by a person of ordinary skill in the art without departing from the scope of the invention as claimed.

What is claimed is:

1. A method of treating or delaying onset of levodopa-induced dyskinesia in an individual, comprising administering to the individual an effective amount of mammalian target of rapamycin (mTOR) inhibitor.

2. The method of claim 1, wherein the mTOR inhibitor is selected from the group consisting of rapamycin, temsirolimus, everolimus, and AP-23573, and combinations thereof.

3. The method of claim 1, wherein the mTOR inhibitor is administered in combination with levodopa or a precursor or functional analogue thereof.

4. The method of claim 1, wherein the mTOR inhibitor is administered in a dosage amount of from about 0.01 mg/kg to about 10 mg/kg.

5. The method of claim 1, wherein the mTOR inhibitor blocks S6 phosphorylation increase produced by administration of levodopa without affecting extracellular signal-regulated kinase (ERK) phosphorylation.

6. The method of claim 1, wherein the individual has levodopa-induced dyskinesia.

7. The method of claim 1, wherein the individual is at risk of developing levodopa-induced dyskinesia.

8. A method of treating or delaying onset of dyskinesia in an individual, wherein the dyskinesia is induced by administration of a drug causing abnormal protein expression in striatal medium-size spiny neurons (MSNs), comprising administering to the individual an effective amount of mammalian target of rapamycin (mTOR) inhibitor.

9. The method of claim 8, wherein the mTOR inhibitor is selected from the group consisting of rapamycin, temsirolimus, everolimus, and AP-23573, and combinations thereof.

10. The method of claim 8, wherein the mTOR inhibitor is administered in combination with a drug causing abnormal protein expression in striatal medium-size spiny neurons (MSNs).

11. The method of claim 8, wherein the mTOR inhibitor is administered in a dosage amount of from about 0.01 mg/kg to about 10 mg/kg.

12. The method of claim 8, wherein the mTOR inhibitor blocks S6 phosphorylation increase produced by administration of a drug causing abnormal protein expression in striatal medium-size spiny neurons (MSNs) without affecting extracellular signal-regulated kinase (ERK) phosphorylation.

13. The method of claim 8, wherein the individual has drug-induced dyskinesia.

14. The method of claim 8, wherein the individual is at risk of developing drug-induced dyskinesia.

15. A combination therapy for an individual having Parkinson's Disease, comprising levodopa or a precursor or functional analogue thereof and mammalian target of rapamycin (mTOR) inhibitor.

16. The combination therapy of claim 15, wherein the mTOR inhibitor is selected from the group consisting of rapamycin, temsirolimus, everolimus, and AP-23573, and combinations thereof.

17. The combination therapy of claim 15, wherein the mTOR inhibitor is provided in a dosage amount of from about 0.01 mg/kg to about 10 mg/kg.

18. The combination therapy of claim 15, wherein the levodopa or precursor or functional analogue thereof is provided in a dosage amount of from about 0.1 mg/kg mg to about 40 mg/kg.

19. The combination therapy of claim 15, wherein the mTOR inhibitor and the levodopa or precursor or functional analogue thereof are provided in a single dosage form.

20. The combination therapy of claim 15, wherein the mTOR inhibitor and the levodopa or precursor or functional analogue thereof are provided in separate dosage forms.
